(19) United States
(12) Patent Application Publication
(10) Pub. No.: US 2005/0288310 A1
(43) Pub. Date: Dec. 29, 2005

(54) Methods of treating cellular proliferative disease using naphthalimide and PARP-1 inhibitors

(75) Inventor: Dennis M. Brown, Menlo Park, CA (US)

Correspondence Address: DORSEY & WHITNEY LLP, 555 CALIFORNIA STREET, SUITE 1000, SUITE 1000, SAN FRANCISCO, CA, 94104, US

(73) Assignee: ChemGenex Pharmaceuticals, Inc.

(21) Appl. No.: 11/146,486

(22) Filed: Jun. 6, 2005

Related U.S. Application Data
(60) Provisional application No. 60/577,101, filed on Jun. 4, 2004.

(51) Int. Cl. A61K 31/522; A61K 31/473; A61K 31/455

(52) U.S. Cl. 514/263.31; 514/296; 514/356

(57) ABSTRACT

A method of treating a cellular proliferative in a patient through the use of a naphthalimide and a PARP-1 inhibitor is disclosed. In a one embodiment, the naphthalimide is amonafide or an analog thereof.

This application claims the benefit of U.S. Provisional Application No.60/577,101, filed Jun. 4, 2004.

### FIELD OF THE INVENTION

The technical field of the invention relates to the use of naphthalimides and poly(ADP-ribose) polymerase-1 inhibitors to treat a cellular proliferative disease.

### BACKGROUND OF THE INVENTION

Amonafide, a naphthalimide analog, is a known antitumor compound. U.S. Pat. No. 6,630,173 and U.S. patent Publication No. 2004/0082788, both of which are expressly incorporated by reference herein.

Although the clinical activity of naphthalimides against cellular proliferative diseases has been established, improvements in the overall efficacy of treatment are desirable.

### SUMMARY OF THE INVENTION

In accordance with the objective outlined above, the present invention provides a method of treating a cellular proliferative disease, comprising administering to a patient in need thereof a naphthalimide and a poly(ADP ribose) polymerase-1 (PARP-1) inhibitor. In a preferred embodiment, the naphthalimide comprises amonafide or an amonafide analog.

In one embodiment, the PARP-1 inhibitor administered to the patient comprises caffeine. In another embodiment, the PARP-1 inhibitor comprises nicotinamide.

In one embodiment, the naphthalimide is administered in combination with the PARP-1 inhibitor. In another embodiment, the naphthalimide is administered sequentially with the PARP-1 inhibitor. In this embodiment, the naphthalimide is administered before the PARP-1 inhibitor is administered or the PARP-1 inhibitor is administered before the naphthalimide.

In one embodiment, the cellular proliferative disease treated by the method is a solid tumor. In a preferred embodiment, the cellular proliferative disease is prostate cancer. In a further preferred embodiment, the cellular proliferative disease is breast cancer.

In one embodiment, the cellular proliferative disease is a solid tumor. In a preferred embodiment, the cellular proliferative disease is prostate cancer. In a further preferred embodiment, the cellular proliferative disease is breast cancer.

In a preferred embodiment, the patient so treated is a human.

The methods of treating cellular proliferative diseases of the present invention have other features and advantages which will be apparent from or are set forth in more detail in the accompanying figures, which are incorporated in and form a part of this specification, and the following Detailed Description of the Invention, which together serve to explain the principles of the present invention.

### DETAILED DESCRIPTION OF THE FIGURES

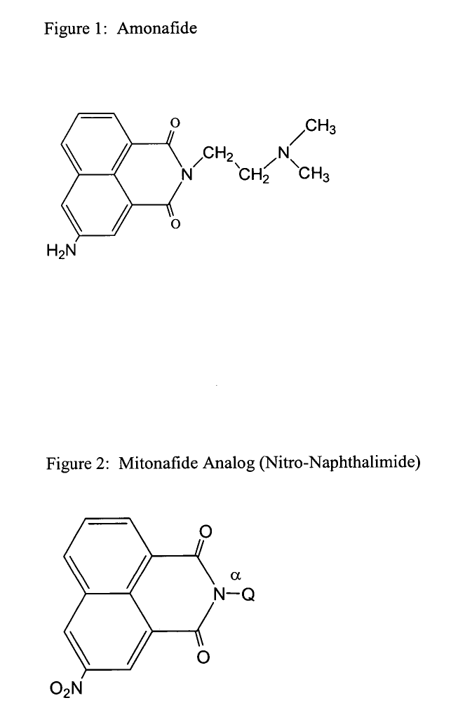

FIG. 1 depicts the structure of the naphthalimide, amonafide.

FIG. 2 depicts the structure of a 3-nitro-naphthalimide or mitonafide analog.

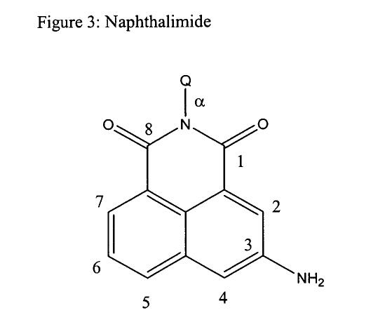

FIG. 3 depicts the structure of a naphthalimide. The Q in the figure represents a substituent group, as described herein.

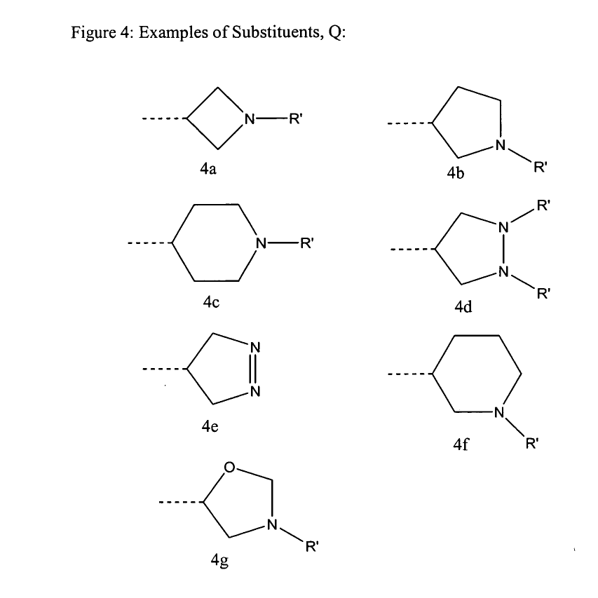

FIG. 4 depicts chemical structures of several possible Q substituent groups that may substitute in the naphthalimide structure of FIG. 3, or in the nitro-naphthalimide structure of FIG. 2. The ring structures each depict a bond to the amide nitrogen. This bond (marked by a dashed line) represents the point of attachment to the naphthalimide structure of FIG. 3 or to the nitro-naphthalimide structure of FIG. 2.

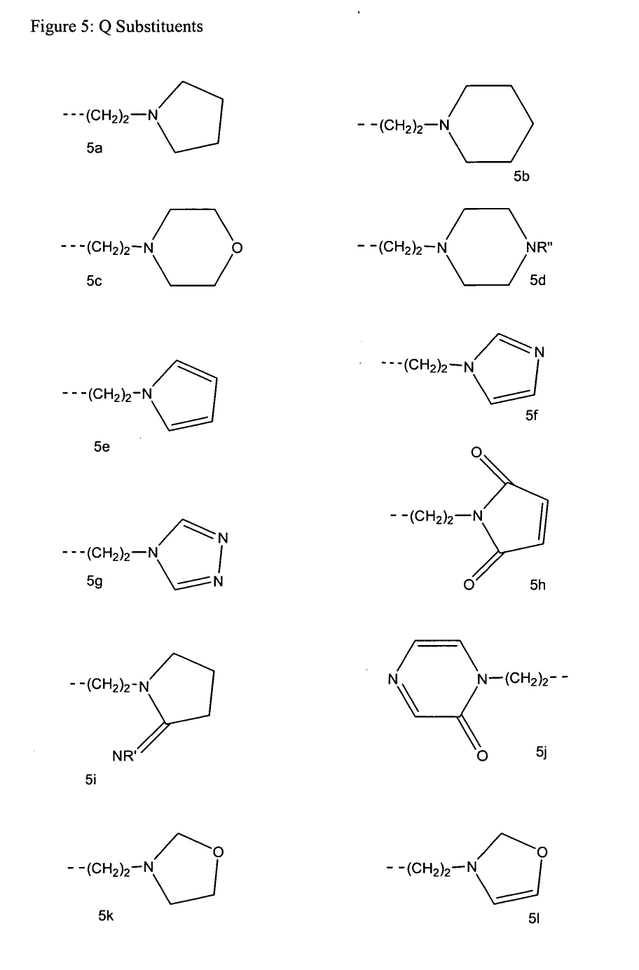

FIG. 5 depicts other groups of possible Q substituent groups. Similar to those of FIG. 4, these groups may substitute for Q in the naphthalimide structure of FIG. 3, or in the nitro-naphthalimide structure of FIG. 2. Each structure depicts a bond (marked by a dashed line), which represents the point of attachment of the substituent group to the naphthalimide structure of FIG. 3 or to the nitro-naphthalimide structure of FIG. 2.

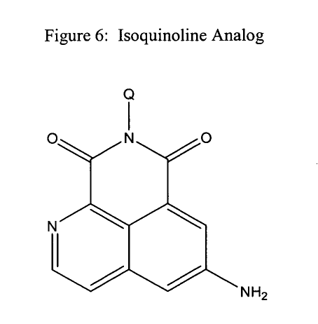

FIG. 6 depicts the structure of an isoquinoline analog of amonafide. The Q in the figure represents a substituent group, as described herein.

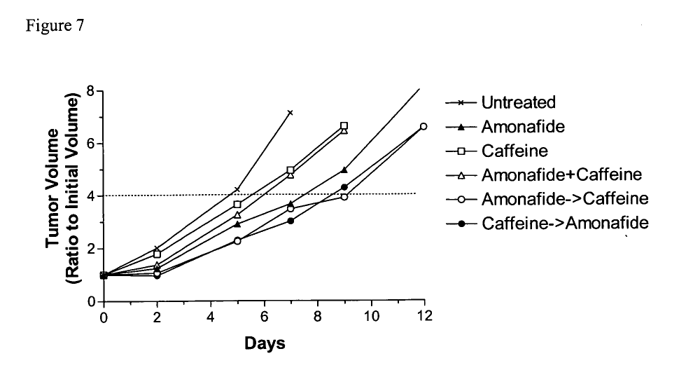

FIG. 7 depicts the tumor growth delay of RIF-1 tumors after treatment with amonafide in combination or sequentially with caffeine.

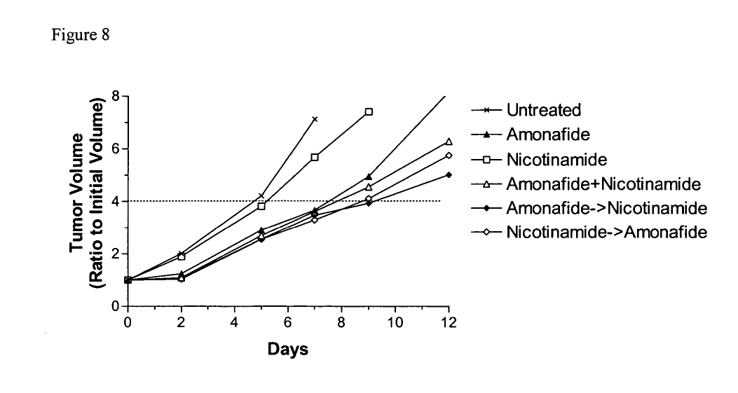

FIG. 8 depicts the tumor growth delay of RIF-1 tumors after treatment with amonafide in combination or sequentially with nicotinamide.

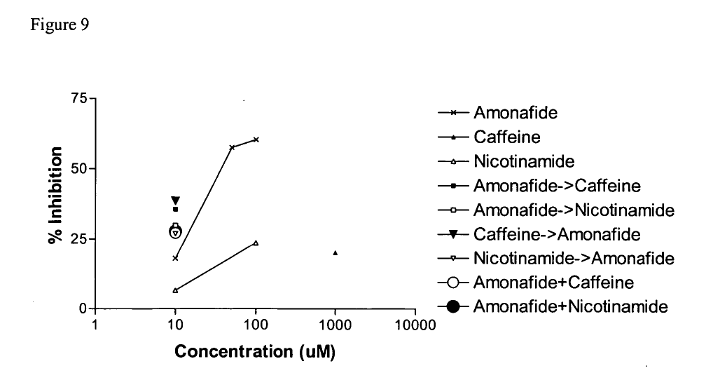

FIG. 9 depicts an increase in inhibition of cellular growth observed after treatment with amonafide in combination or sequentially with nicotinamide or caffeine in an in vitro cell viability assay.

### DETAILED DESCRIPTION OF THE INVENTION

Naphthalimides are known to be effective in the treatment of cellular proliferative diseases such as cancers. As used herein, naphthalimide includes all members of that chemical family including benz isoquinoline dione and analogs thereof. Naphthalimides have the structure set forth in FIG. 3. In addition, the naphthalimide includes amonafide such as set forth in FIG. 1 and analogs thereof. Naphthalimide also includes nitro-naphthalimide, e.g., mitonafide as set forth in FIG. 2 and analogs such as isoquinoline analogs such as set forth in FIG. 6. It should be understood that Q in each of the FIGS. 2 and 6 correspond to the structure set forth in FIG. 4 as well as other substituents at Q.

The present invention is based on the discovery that poly(ADP ribose) polymerase-1 (PARP-1) inhibitors increase the efficacy of naphthalimide in the treatment cellular proliferative diseases. For example, as disclosed herein, the increase in tumor growth delay observed after treatment with amonafide alone is increased if the tumor is also treated with a PARP-1 inhibitor.

Accordingly, it is one aspect of the invention to provide for a method of treating a cellular proliferative disease comprising administering to a patient in need thereof a naphthalimide and PARP-1 inhibitor.

Poly(ADP ribose) polymerase-1 (PARP-1) functions as a DNA damage sensor and signaling molecule. Inhibition of PARP-1 has proven useful for radiosensitization of cancer cells and inhibition of DNA repair by alkylating agents. Examples of PARP-1 inhibitors include, but are not limited to, caffeine, nicotinamide, 3 aminobenzamide, 4-Amino-1,8-naphthalimide, 6(5H)-Phenanthridinone, 5-Aminoisoquinolinone (5-AIQ), Hydrochloride, 4-Hydroxyquinazoline, 4-Quinazolinol, 1,5-Isoquinolinediol, 5-Hydroxy-1(2H)-isoquinolinone, 3,4-Dihydro-5-[4-(1-piperidinyl)butoxy]-1(2H)-isoquinolinone (DPQ). (Southan, G. J. and Csaba S., Current Medicinal Chemistry, 10: 321-340 (2003), incorporated by reference herein.) In a preferred embodiment, caffeine and amonafide are administered to a patient to treat a cellular proliferative disease. In further preferred embodiment, nicotinamide and amonafide are administered to a patient to treat a cellular proliferative disease.

A patient for the purposes of the present invention includes both humans and other animals, particularly mammals, and organisms, in need of treatment for a cellular proliferative disease. Thus the methods are applicable to both human therapy and veterinary applications. In the preferred embodiment the patient is a mammal, and in the most preferred embodiment the patient is human.

Cellular proliferative diseases that can be treated by the compounds of the invention include, for example, psoriasis, skin cancer, viral induced hyperproliferative HPV-papiloma, HSV-shingles, colon cancer, bladder cancer, breast cancer, melanoma, ovarian carcinoma, prostate cancer, or lung cancer, and a variety of other cancers as well.

According to a preferred embodiment, the cellular proliferative disease is a tumor, e.g., a solid tumor. Solid tumors that are particularly amenable to treatment by the claimed methods include carcinomas and sarcomas. Carcinomas include those malignant neoplasmas derived from epithelial cells which tend to infiltrate (invade) the surrounding tissues and give rise to metastases. Adenocarcinomas are carcinomas derived from glandular tissue or in which the tumor cells form recognizable glandular structures. Sarcomas broadly include tumors whose cells are embedded in a fibrillar or homogeneous substance like embryonic connective tissue. It will be understood that the method of the invention is not limited to the treatment of these tumor types, but extends to any solid tumor derived from any organ system.

In one aspect of the invention, the naphthalimide is administered in combination with a PARP-1 inhibitor. By “in combination with” is meant that the compounds are administered to the patient at the same time. According to one embodiment, the compounds are administered in a single dosage form. In another embodiment, the compounds are administered as separate doses.

Another aspect of the invention provides for the sequential administration of the naphthalimide and PARP-1 inhibitor. For example, administration of a naphthalimide may be followed by administration of a PARP-1 inhibitor; or administration of a PARP-1 inhibitor may be followed by administration of a naphthalimide.

When administration of the two compounds is sequential, a defined length of time separates administration of the two compounds. According to one embodiment, administration of each compound is separated by at least about 5 minutes but by no more than 8 hours. Generally, when administration of the two compounds is sequential, the time separating the administration of each compound is no more than two plasma half lives of the first administered compound. According to a preferred embodiment, administration of each compound is separated by about 30 minutes. According to another embodiment, administration of each compound is separated by about 1 hour. According to another embodiment, administration of each compound is separated by about 2 hours. In yet a further embodiment, administration of each compound is separated by about 3 hours. In yet a further embodiment, administration of each compound is separated by about 4 hours.

The optimal time separating the administration of the compounds will vary depending on the dosage used, the clearance rate of each compound, and the particular patient treated. According to the claimed methods, the naphthalimide and the PARP-1 inhibitor used are administered such that the compounds are present together in the patient's system in active form during the treatment of the patient. That is, the compound that is administered first will be present in the patient in an active form after the second compound is administered.

In yet another aspect of the invention, the naphthalimide and the PARP-1 inhibitor are administered with an antiproliferative agent. As used herein, antiproliferative agents are compounds which induce cytostasis or cytotoxicity. “Cytostasis” is the inhibition of cells from growing while “cytotoxicity” is defined as the killing of cells. Specific examples of antiproliferative agents include: antimetabolites, such as methotrexate, 5-fluorouracil, gemcitabine, cytarabine, pentostatin, 6-mercaptopurine, 6-thioguanine, L-asparaginase, hydroxyurea, N-phosphonoacetyl-L-aspartate (PALA), fludarabine, 2-chlorodeoxyadenosine, and floxuridine; structural protein agents, such as the vinca alkaloids, including vinblastine, vincristine, vindesine, vinorelbine, paclitaxel, and colchicine; agents that affect NF-κB, such as curcumin and parthenolide; agents that affect protein synthesis, such as homoharringtonine; antibiotics, such as dactinomycin, daunorubicin, doxorubicin, idarubicin, bleomycins, plicamycin, and mitomycin; hormone antagonists, such as tamoxifen and luteinizing hormone releasing hormone (LHRH) analogs; nucleic acid damaging agents such as the alkylating agents mechlorethamine, cyclophosphamide, ifosfamide, chlorambucil, dacarbazine, methylnitrosourea, semustine (methyl-CCNU), chlorozotocin, busulfan, procarbazine, melphalan, carmustine (BCNU), lomustine (CCNU), and thiotepa, the intercalating agents doxorubicin, dactinomycin, daurorubicin and mitoxantrone, the topoisomerase inhibitors etoposide, camptothecin and teniposide, and the metal coordination complexes cisplatin and carboplatin.

A chemical compound is a “chemopotentiator” when it enhances the effect of another compound in a more than additive fashion relative to the activity of the chemopotentiator or other compound used alone. In some cases, a “chemosensitizing” effect may be observed. This is defined as the effect of use of compound that if used alone would not demonstrate significant antitumor effects but would improve the antitumor effects of an antiproliferative agent in a more than additive fashion than the use of the antiproliferative agent by itself.

The compounds may be provided in a range of concentrations, depending on the cellular proliferative disease to be treated, host species, clearance rate of each compound, drug absorption, bioavailability, mode of administration.

Naphthalimides are provided in a dosage amount sufficient to modulate a cellular proliferative disease. In one embodiment, modulation of a cellular proliferative disease comprises a reduction in tumor growth. In another embodiment, modulation of a disease comprises inhibition of tumor growth. In another embodiment, modulation of a cellular proliferative disease comprises an increase in tumor volume quadrupling time (described below). In another embodiment, modulation of a cellular proliferative disease comprises a chemopotentiator effect. In another embodiment, modulation of a disease comprises a chemosensitizing effect. In other embodiments, modulation of a disease comprises cytostasis. In still other embodiments, modulation of a disease comprises a cytotoxic effect.

In a preferred embodiment, modulation of a cellular proliferative is determined by measuring the tumor volume quadrupling time of a tumor. Tumor volume quadrupling time as used herein means the time (days) for treated and untreated tumors to grow to four times (4×) their initial treatment volume.

In a preferred embodiment, a naphthalimide is provided for administration at between about 1-1000 mg/kg. In a further preferred embodiment, a naphthalimide is provided for administration at between about 20-200 mg/kg. In yet a further preferred embodiment, a naphthalimide is provided for administration at between about 30-100 mg/kg. Generally the concentration of naphthalimide will depend on the NAT-2 genotype of the patient. See for example, U.S. Ser. No. 11/048,614, incorporated by reference herein.

PARP-1 inhibitors are provided in a dosage amount sufficient to modulate the efficacy of the naphthalimide. In a preferred embodiment, the PARP-1 inhibitor is provided in an amount sufficient to increase the reduction in tumor growth as compared to treatment with amonafide alone. In another embodiment, modulation of the efficacy of amonafide comprises increased inhibition of tumor growth as compared to treatment with amonafide alone. In another embodiment, modulation of the efficacy of amonafide comprises increase in tumor volume quadrupling time as compared to treatment with amonafide alone. In another embodiment, modulation of the efficacy of amonafide comprises an increased chemopotentiator effect as compared to treatment with amonafide alone. In another embodiment, modulation of the efficacy of amonafide comprises an increased chemosensitizing effect as compared to treatment with amonafide alone. In other embodiments, modulation of the efficacy of amonafide comprises increased cytostasis as compared to treatment with amonafide alone. In still other embodiments, modulation of the efficacy of amonafide comprises increased cytotoxic effect as compared to treatment with amonafide alone.

In a preferred embodiment, a PARP-1 inhibitor is administered at between 1-2000 mg/kg. In a preferred embodiment, a PARP-1 inhibitor is provided for administration at between about 20-1500 mg/kg. In yet a further preferred embodiment, a PARP-1 inhibitor is provided for administration at between about 50-1000 mg/kg. In yet a further preferred embodiment, a PARP-1 inhibitor is provided for administration at between about 75-500 mg/kg.

Methods of Administration

The compositions may be administered by any method including oral, rectal, topical (including transdermal devices, aerosols, creams, ointments, lotions, and dusting powders), parenteral (including subcutaneous, intramuscular, and intravenous), ocular (ophthalmic), pulmonary (nasal or buccal inhalation), or nasal administration; although the most suitable route in any given case will depend largely on the nature and severity of the condition being treated and on the nature of the active ingredient.

In one embodiment, the compounds are administered orally, for example in tablet form, or by inhalation, for example in aerosol or other atomisable formulations or in dry powder formulations, using an appropriate inhalation device such as those known in the art. The compounds of the invention may also be administered intranasally.

In the case of oral delivery, the dosage form would allow that suitable concentrations of a naphthalimide would be provided in a form such that an adequate plasma level could be achieved to provide the chemopotentiation of the other chemotherapeutic compound(s). Tablets, capsules, suspensions or solutions may contain 10 milligrams to 2 grams per dose treatment to achieve the appropriate plasma concentrations.

A compound may be combined as the active ingredient in intimate admixture with a pharmaceutical carrier according to conventional pharmaceutical compounding techniques. The carrier may take a wide variety of forms depending on the nature of the preparation desired for administration, i.e., oral, parenteral, etc. In preparing oral dosage forms, any of the usual pharmaceutical media may be used, such as water, glycols, oils, alcohols, flavoring agents, preservatives, coloring agents, and the like in the case of oral liquid preparations (e.g., suspensions, elixirs, and solutions); or carriers such as starches, sugars, microcrystalline cellulose, diluents, granulating agents, lubricants, binders, disintegrating agents, etc. in the case of oral solid preparations such as powders, capsules, and tablets. Solid oral preparations are preferred over liquid oral preparations. Because of their ease of administration, tablets and capsules are the preferred oral dosage unit form. If desired, capsules may be coated by standard aqueous or non-aqueous techniques.

In addition to the dosage forms described above, the compounds of the invention may be administered by controlled release means and devices.

The compounds of the invention may also be administered via ocular methods, for example via ophthalimic inserts. Ophthalmic inserts are made from compression molded films which are prepared on a Carver Press by subjecting the powdered mixture of active ingredient and HPC to a compression force of 12,000 lb. (gauge) at 149.degree. C. for 1-4 min. The film is cooled under pressure by having cold water circulate in the platen. The inserts are then individually cut from the film with a rod-shaped punch. Each insert is placed in a vial, which is then placed in a humidity cabinet (88% relative humidity at 30.degree. C.) for 2-4 days. After removal from the cabinet, the vials are capped and then autoclaved at 121.degree. C. for 0.5 hr.

The inhalable form may be, for example, an atomisable composition such as an aerosol comprising the compounds of the invention in solution or dispersion in a propellant or a nebulizable composition comprising a dispersion of the compound of the invention in an aqueous, organic or aqueous/organic medium, or a finely divided particulate form comprising the compounds of the invention in finely divided form optionally together with a pharmaceutically acceptable carrier in finely divided form.

The compositions containing a compound of this invention may also comprise an additional agent selected from the group consisting of cortiocosteroids, bronchodilators, antiasthmatics (mast cell stabilizers), anti-inflammatories, antirheumatics, immunosuppressants, antimetabolites, immunomodulators, antipsoriatics, and antidiabetics. Specific compounds include theophylline, sulfasalazine and aminosalicylates (anti-inflammatories); cyclosporin, FK-506, and rapamycin (immunosuppressants); cyclophosphamide and methotrexate (antimetabolites); and interferons (immunomodulators).

An aerosol composition suitable for use as the inhalable form may comprise the compounds of the invention in solution or dispersion in a propellant, which may be chosen from any of the propellants known in the art. Suitable such propellants include hydrocarbons such as n-propane, n-butane or isobutane or mixtures of two or more such hydrocarbons, and halogen-substituted hydrocarbons, for example fluorine-substituted methanes, ethanes, propanes, butanes, cyclopropanes or cyclobutanes, particularly 1,1,1,2-tetrafluoroethane (HFA134a) and heptafluoropropane (HFA227), or mixtures of two or more such halogen-substituted hydrocarbons. Where the compounds of the invention are present in dispersion in the propellant, i.e. where present in particulate form dispersed in the propellant, the aerosol composition may also contain a lubricant and a surfactant, which may be chosen from those lubricants and surfactants known in the art. The aerosol composition may contain up to about 5% by weight, for example 0.002 to 5%, 0.01 to 3%, 0.015 to 2%, 0.1 to 2%, 0.5 to 2% or 0.5 to 1%, by weight of the compounds of the invention, based on the weight of the propellant. Where present, the lubricant and surfactant may be in an amount up to 5% and 0.5% respectively by weight of the aerosol composition. The aerosol composition may also contain ethanol as co-solvent in an amount up to 30% by weight of the composition, particularly for administration from a pressurized metered dose inhalation device.

A finely divided particulate form, i.e. a dry powder, suitable for use as the inhalable form may comprise the compounds of the invention in finely divided particulate form, optionally together with a finely divided particulate carrier, which may be chosen from materials known as carriers in dry powder inhalation compositions, for example saccharides, including monosaccharides, disaccharides and polysaccharides such as arabinose, glucose, fructose, ribose, mannose, sucrose, lactose, maltose, starches or dextran. As especially preferred carrier is lactose. The dry powder may be in capsules of gelatin or plastic, or in blisters, for use in a dry powder inhalation device, preferably in dosage units of 5 .mu.g to 40 mg of the active ingredient. Alternatively, the dry powder may be contained as a reservoir in a multi-dose dry powder inhalation device.

In the finely divided particulate form, and in the aerosol composition where the compounds are present in particulate form, the compound may have an average particle diameter of up to about 10 nanometers, for example 1 to 5 nanometers. The particle size of the compound of the invention, and that of a solid carrier where present in dry powder compositions, can be reduced to the desired level by conventional methods, for example by grinding in an air-jet mill, ball mill or vibrator mill, microprecipitation, spray-drying, lyophilisation or recrystallisation from supercritical media.

The inhalable medicament comprising the pharmaceutical compositions of the invention may be administered using an inhalation device suitable for the inhalable form, such devices being well known in the art. Accordingly, the invention also provides a pharmaceutical product comprising the compounds of the invention in inhalable form as hereinbefore described in association with an inhalation device. In a further aspect, the invention provides an inhalation device containing the compounds of the invention in inhalable form as hereinbefore described.

Where the inhalable form is an aerosol composition, the inhalation device may be an aerosol vial provided with a valve adapted to deliver a metered dose, such as 10 to 100 .μl, e.g. 25 to 50 μl, of the composition, i.e. a device known as a metered dose inhaler. Suitable such aerosol vials and procedures for containing within them aerosol compositions under pressure are well known to those skilled in the art of inhalation therapy. Where the inhalable form is a nebulizable aqueous, organic or aqueous/organic dispersion, the inhalation device may be a known nebulizer, for example a conventional pneumatic nebulizer such as an airjet nebulizer, or an ultrasonic nebulizer, which may contain, for example, from 1 to 50 mL, commonly 1 to 10 mL, of the dispersion; or a hand-held nebulizer such as an AERX (ex Aradigm, US) or BINEB (Boehringer Ingelheim) nebulizer which allows much smaller nebulized volumes, e.g. 10 to 100 .mu.l, than conventional nebulizers. Where the inhalable form is the finely divided particulate form, the inhalation device may be, for example, a dry powder inhalation device adapted to deliver dry powder from a capsule or blister containing a dosage unit of the dry powder or a multidose dry powder inhalation device adapted to deliver, for example, 25 mg of dry powder per actuation. Suitable such dry powder inhalation devices are well known.

Pharmaceutical Compositions

Another aspect of the invention provides for pharmaceutical compositions comprising a naphthalimide and a PARP-1 inhibitor. The naphthalimide and PARP-1 inhibitor may be in intimate admixture or they may isolated from each other. In some embodiments, the naphthalimide in the pharmaceutical compositions is a pharmaceutically acceptable salt. Accordingly, pharmaceutical compositions may contain pharmaceutically acceptable carriers and, optionally, other therapeutically active ingredients.

The compositions include compositions suitable for oral, rectal, topical (including transdermal devices, aerosols, creams, ointments, lotions, and dusting powders), parenteral (including subcutaneous, intramuscular, and intravenous), ocular (ophthalmic), pulmonary (nasal or buccal inhalation), or nasal administration; although the most suitable route in any given case will depend largely on the nature and severity of the condition being treated and on the nature of the active ingredient. The agents may be conveniently presented in unit dosage form and prepared by any of the methods well known in the art of pharmacy.

Additives, carriers or excipients are well known in the art, and are used in a variety of formulations. See for example, Gilman, A. G. et al., eds., THE PHARMACOLOGICAL BASIS OF THERAPEUTICS, 8^{th} Ed. Pergamon Press, New York, (1990), incorporated herein by reference in its entirety. In practical use, the compositions of the invention can be combined as the active ingredient in intimate admixture with a pharmaceutical carrier or excipient according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols (e.g., polyethylene glycol), oils, alcohols, flavoring agents, sweeteners, preservatives, coloring agents and the like in the case of oral liquid preparations, such as, for example, suspensions, elixirs and solutions; or carriers such as starches (e.g. corn or other), sugars, lactose, serum albumin, microcrystalline cellulose, buffers (e.g., sodium acetate), diluents, granulating agents, lubricants, binders, disintegrating agents and the like in the case of oral solid preparations such as, for example, powders, hard and soft capsules and tablets, with the solid oral preparations being preferred over the liquid preparations.

i. Oral Dosage Forms

Because of their ease of administration, tablets and capsules represent a particularly advantageous oral dosage unit form in which case solid pharmaceutical carriers are obviously employed. If desired, tablets may be coated by standard aqueous or nonaqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

ii. Liquid Dosage Forms

The pharmaceutical compositions can be provided in liquid dosage forms.

In one embodiment, the liquid dosage form prepared according to the methods of the invention is a stable sterile aqueous solution of a naphthalimide or naphthalimide salt (e.g., amonafide or amonafide dihydrochloride) in a sealed container such as an ampoule or vial, is in unit dosage form suitable for intravenous administration, has a concentration of a naphthalimide or naphthalimide salt between about 1 and about 250 mg/mL, and has a pH between about 3.0 and 7.0. In a preferred embodiment, the concentration of a naphthalimide or naphthalimide salt is about 20 mg/mL.

In a preferred embodiment, the pH of the liquid dosage form is about 6.0. Preferably, the pH is adjusted, if necessary, using a nontoxic, pharmaceutically and therapeutically acceptable inorganic source base. In a preferred embodiment, the base is a mineral base. In a more preferred embodiment, the base is sodium hydroxide.

In one embodiment, the liquid dosage form prepared according to the methods of the invention preferably is free of any other added chemicals. In another embodiment, the liquid dosage form contains a customary, physiologically acceptable excipient or carrier such as a preservative or tonicity agent. In one embodiment, an aqueous solution of amonafide comprises a carrier or excipient. Preferably, a carrier or excipient, when provided, is present at a concentration between about 0.1 mg/ml to 100 mg/ml.

According to one embodiment, the liquid dosage form is stable. “Stable” means that the liquid dosage form exhibits less than 5% loss of potency as measured by high performance liquid chromatography (HPLC) upon storage for 1 month at 60° C. or 9 months 40° C.

The compositions of the invention may be conveniently presented in unit dosage forms, and prepared by any methods known in the art of pharmacy. The term “unit dosage form” refers to physically discrete units suitable as unitary dosages for human subjects and other mammals, each unit containing a predetermined quantity of active material calculated to produce the desired therapeutic effect in association with a suitable pharmaceutical excipient or carrier.

In the preferred embodiment, the pharmaceutical compositions are water soluble, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

Synthesis

The pharmaceutical compositions of the invention may be synthesized using known techniques. According to one embodiment, the naphthalimide used in the present invention is amonafide synthesized according to a method disclosed in U.S. Publication No. 2004/0082788, published Apr. 29, 2004, hereby incorporated by reference in its entirety.

i. Mitonafide Analogs

Nitro-naphthalimide, or a “mitonafide analog”, as indicated in the structure in FIG. 2 is made by adding an aliphatic diamine to 3-nitro-1,8,-naphthalic anhydride in an organic solvent mixture, and refluxing to obtain the nitro naphthalimide. A general scheme depicting the reaction is below:

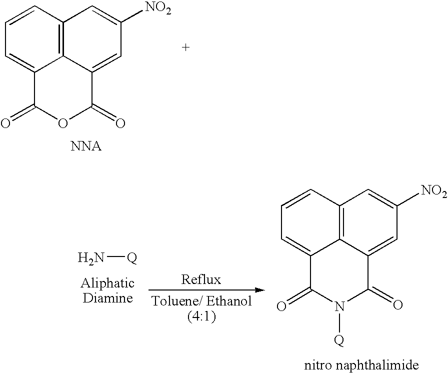

The aliphatic diamine used in the synthesis of a nitro naphthalimide can vary. The choice of aliphatic diamine allows synthesis of a mitonafide analog with, for example, a carbon chain length of 1-6. According to a preferred embodiment, the aliphatic diamine used is N,N-dimethylethylenediamine.

The structure in FIG. 2 indicates a substituent group, Q. Q may represent a variety of structures, including those indicated in FIGS. 4 and 5.

One class of compounds synthesized by the methods of this invention includes naphthalimides with the structure depicted in FIG. 3. The group Q in FIG. 3 may be a variety of substituents, for example, the groups represented in FIG. 4. For example, Q may be 1-R′-azetid-3-yl (FIG. 4a), 1-R′-pyrrolid-3-yl (FIG. 4b), 1-R′-piperid-4-yl (FIG. 4c), 1,2-diR′-1,2-diazolid-4-yl (FIG. 4d), 1,2-diazol-1-en4yl (FIG. 4e), 1-R′-piperid-3-yl (FIG. 4f), 3-R′-oxazolid-5-yl (FIG. 4g). In FIG. 4, R′=alkyl, unsaturated alkyl, acyl, alkoxy, aryl, amino, substituted amino, sulfo, sulfamoyl, carboxy, carbamyl, cyano.

In another embodiment, the structure in FIG. 3 represents a naphthalimide of the invention, wherein Q represents —(CH₂)₂NR₂, wherein R=methyl, ethyl, propyl, butyl, etc. NR₂ in this representation may represent a heterocyclic group. Thus, Q may be any one of the groups shown in FIG. 5.

In some embodiments, R₂═—(CH₂)n- where n=2 to 6, or R₂═—(CH₂)mX—(CH₂)n— where m and n can be 0 to 5 and X can be NR″ (where R″=hydrogen, alkyl, unsaturated alkyl, acyl, alkoxy, aryl, amino, substituted amino, sulfo, sulfamoyl, carboxy, carbamyl, cyano, or is not present), O, or S. Furthermore, these cyclic groups may have unsaturated bonds and may also bear substituents such as alkyl, aryl, or heteroaryl.

Further examples of the substituent group Q include, for example, those shown in FIG. 5, which are 1-pyrrolidyl (5a), 3-R′-1-piperidyl (FIG. 5b), morpholino (FIG. 5c), 1-R′-piperazin4-yl (FIG. 5d), 1-pyrrolyl (FIG. 5e), 1-imidazolyl (FIG. 5f), 1,3,5-triazol-1-yl (FIG. 5g), N-maleimido (FIG. 5h), 2-(R′-imino)pyrrolidyl (FIG. 5i), pyrazin-2-on-1-yl (FIG. 5j), 3-oxazolidyl (FIG. 5k), 3-oxazolyl (FIG. 5l), and others known in the art, for example, 2-pyrrolyl, 3-chloro-1-pyrrolidyl, 2-nitro-1-imidazolyl, 4-methoxy-1-imidazolyl, 3-methyl-1-imidazolyl. In the structures depicted in FIG. 5, R′=alkyl, unsaturated alkyl, acyl, alkoxy, aryl, amino, substituted amino, sulfo, sulfamoyl, carboxy, carbamyl, cyano, and other functional groups known to those skilled in the art.

Another group of compounds of the invention are naphthalimides having an amino group attached to other positions in the naphthalimide rings. According to one embodiment, the naphthalimide ring is modified to include one or more amino groups at positions other than position 3 of the naphthalimide ring. According to another embodiment, the naphthalimide ring is modified to include one or more amino groups at positions in addition to the amino group at position 3 of the naphthalimide ring. In another embodiment, the amino group at position 3 is replaced with a substituent group. Examples of such groups include: alkyl, aryl, nitro, substituted amino, sulfamoyl, halo, carboxy, carbamyl, cyano, and other functional groups known to those skilled in the art. In yet another embodiment, an additional group is attached to the naphthalimide ring also comprising an amino group at position 3. Examples of such substituent groups include: alkyl, aryl, nitro, amino, substituted amino, sulfamoyl, halo, carboxy, carbamyl, cyano, and other functional groups known to those skilled in the art.

Alternatively, the amino group at position 3 may be replaced by other substituent groups. Examples of substituent groups include: alkyl, aryl, nitro, substituted amino, sulfamoyl, halo, carboxy, carbamyl, cyano, and other functional groups known to those skilled in the art.

The naphthalene ring can be replaced with one bearing one or more nitrogen atoms in either or both rings. An example would be isoquinoline analogs (FIG. 6), where Q is as previously defined. A preferred isoquinoline analog of amonafide is where Q is —(CH₂)n-N(CH₃)₂, where n is 1-12 or more. In a more preferred embodiment, n is 1-6. The isoquinoline analog may also have one or more substituent groups (as described herein for other analogs) reducing one or more hydrogens of the methyl and/or methylene groups.

An organic solvent is used in the method of the invention for refluxing the aliphatic diamine and 3-nitro-1,8,-naphthalic anhydride. In one embodiment, the organic solvent is ethanol. In another embodiment, the organic solvent is dimethylformamide. In yet another embodiment, the organic solvent is toluene-ethanol. In a preferred embodiment, the organic solvent is toluene-ethanol in a 4:1 ratio.

The mixture is refluxed and monitored, for example, by thin-layer chromatography. Refluxing is performed according to one embodiment for 30 minutes. The resulting mixture is filtered and evaporated to obtain a brown solid of mitonafide or a mitonafide analog.

Each of these naphthalimides may be converted into a mono or diammonium salt as discussed infra.

ii. Naphthalimides

According to another embodiment, the invention includes a method of synthesis of a naphthalimide. In a preferred embodiment, the naphthalimide is amonafide (See Example 2). Amonafide is also known as 5-amino-2-[(dimethylamine)ethyl]-1H-benz[de-]isoquinoline-1,3-(2H)-dione.

The method of naphthalimide synthesis involves dissolving mitonafide or a mitonafide analog in an organic solvent. The organic solvent, according to one embodiment, is dichloromethane-methanol. In a preferred embodiment, dichloromethane-methanol is used in a ratio of 4:1 at 25 mL/g mitonafide.

The method of naphthalimide synthesis further involves adding a reducing agent (e.g., ammonium formate) to the dissolved mitonafide or mitonafide analog together with a catalyst. A variety of reducing agents suitable for reduction of the 3-nitro group are known in the art, including hydrazine, tetralin, ethanol, ascorbic acid, formic acid, formate salts, and phosphinic acid (see, e.g., Johnstone, R. A. W. et al., Chemical Reviews 85 (2) 129 (1985); Entwhistle, I. D. et al., J. C. Soc. Perkin Trans. 1,443(1977)). According to a preferred embodiment, the reducing agent is ammonium formate. Other formate salts include substituted ammonium formates such as 2-hydroxyethylmethyl ammonium formate, methyl ammonium formate and morpholinium. According to a preferred embodiment, 4.5 mol equivalents of ammonium formate are used.

The method of naphthalimide synthesis involves use of a catalyst. A variety of suitable catalysts are known in the art, including the noble metals Pd, Pt, Rh and Raney Nickel (see, e.g., Johnstone, R. A. W. et al. (1985), supra, and Entwhistle, I. D. et al. (1977), supra). In one embodiment, the catalyst is palladium-carbon. In a preferred embodiment, 10% palladium-carbon (about 20% mitonafide weight) is added. The catalyst is mixed at room temperature under nitrogen for about 1 hour. The method further involves filtering the mixture and adding the mixture to a cool water bath (<10° C.) to precipitate. After filtration, a precipitate forms which is dried to yield a naphthalimide, for example, amonafide (C₁₆J₁₇N₃O₂).

iii. Naphthalimide Salts

A further embodiment of the invention includes methods of synthesis of naphthalimide diammonium salts.

In general, naphthalimides are dibasic compounds containing at least two amines and in most cases an amine group covalently linked to an aromatic group. When in contact with an acid, at least one or two of the amines within the naphthalimide may be protonated by reaction with an inorganic or an organic acid to form salts. Such salts are generally weak acids comprising primary, secondary or tertiary ammonium ions formed by protonation of an amine within the amonifide molecule. The counter-ions for such ammonium ions can be any appropriate anion capable of being used in a pharmaceutical composition. In some embodiments, the acidic salts are formed by reacting the naphthalimide with a mineral (inorganic) or organic acid. Such mineral acids include hydrochloric acid, hydrobromic, acid, sulfuric acid, nitric acid and phosphoric acid. Some organic acids which may be used in forming salts of modified include acetic acid, proprionic acid, glycolic acid, pyruvic acid, oxalic acid, maleic acid, malic acid, malonic acid, succinic acid, hydroxy succinic acid, fumaric acid, tartaric acid, citric acid, benzoic acid, cinnamic acid, mandelic acid, methanesulfonic acid, ethanesulfonic acid, p-toluenesulfonic acid and salicylic acid. Inorganic acids include hydrochloric acid, hydrobromic, acid, sulfuric acid, nitric acid and phosphoric acid.

In most embodiments, two amines in the naphthalimide will be protonated to form a diammonium salt. In preferred embodiments, at least 1.5, more preferably 1.75, still more preferably 1.9, still more preferably 1.95, still more preferably 1.99, and most preferably 2.0 mol equivalents of the two amines in the amonafide are protonated. In some instances where more than two amines are present, the possibility exists for protonation of all, or a portion of all, of the amines. For example, if three amines are present, least 1.5, more preferably 1.75, still more preferably 1.9, still more preferably 2.0, still more preferably 2.5, still more preferably 2.75, still more preferably 2.9, still more preferably 2.95, still more preferably 2.99, and most preferably 3.0 mol equivalents of the three amines are protonated.

In one embodiment, the amines of the naphthalimide have similar pK's for protonation. Upon titration of the free base amines of this embodiment with an acid, the amines are similarly protonated during the range of titration. In a preferred embodiment, at least two of the amines of the naphthalimide are greater than 50% protonated, more preferably greater than 75% protonated, still more preferably greater than 90% protonated, still more preferably greater than 95% protonated, still more preferably greater than 99% protonated, and most preferably 100% protonated.

In a further embodiment of the invention, the amines of the naphthalimide have different pK's for protonation. Upon titration of the amines with an acid, the amines will become protonated in a multiphasic manner according to their pK's. For example, the amine that has a higher pK value will become protonated before the amine that has a lower pK value when the free acid form of the naphthalimide is titrated with an acid. In a preferred embodiment, at least one of the amines of the naphthalimide is protonated and at least one of the amines of the naphthalimide is subsequently protonated, preferably greater than 50% protonated, more preferably greater than 75% protonated, still more preferably greater than 85% protonated, still more preferably greater than 95% protonated, still more preferably greater than 99% protonated and most preferably 100% protonated.

Diammonium salts of naphthalimide generally refers to naphthalimide salts which contain two protonated amines with the naphthalimide structure. Partial diammonium salts include those naphthalimides wherein at least 1.5 mol equivalents of the amines are protonated. In some embodiments, the counter-ions may be a mixture of one or more of the base forms of the aforementioned inorganic and/or organic acids.

In a preferred embodiment, the naphthalimide diammonium salt is amonafide dihydrochloride.

According to this embodiment, HCI gas is bubbled over amonafide solution to precipitate a salt form of amonafide. The process is robust and easy to scale up. Amonafide monohydrochloride as disclosed by U.S. Pat. No. 5,420,137 is manufactured by reaction with calculated amount of HCl solution. This process may result inaccurate amount of HCI in the final product and this process is not easy to scale up.

Dihydrochloride salt is more acidic and more soluble in water, as compared to a monohydrochloride salt. As a result, a wider range of drug concentration can be achieved to facilitate further manufacturing process such as lyophilization and more flexible to meet clinical needs.

A preferred embodiment of the present invention provides an improved synthesis of amonafide dihydrochloride salt exhibiting a well-defined crystalline structure with a narrow melting temperature range. The characteristic physical and chemical properties and stability of this form improve the safe handling of this cytotoxic drug during the manufacture of pharmaceutical dosage forms such as oral products including tablet and capsule forms, as well as a wide range of injectable dosage forms, such as liquid or lyophilized forms.

The creation of mono and diammonium salt forms enables the generation of pharmaceutically relevant dosages useful for the treatment of aberrant cell conditions such as hyperproliferative diseases, including, for example, cancer and precancerous conditions.

### EXAMPLES

### Example 1

The effects of the PARP-1 inhibitors, caffeine and nicotinamide, on the efficacy of amonafide treatment in a murine tumor growth delay model were determined as discussed below.

Experimental Details

Compounds:

Naphthalimide-Amonafide dihydrochloride (Quinamed®, AMF), substituted naphthalimide soluble in saline at therapeutic concentrations.

PARP-1 inhibitors—caffeine and nicotinamide.

In vivo Model:

C3H mice were inoculated subcutaneously in the flank with 2×10⁵ radiation-induced fibrosarcoma cells (RIF-1) to produce experimental tumors. When tumors reached ˜100 mm³, test compounds (100 uL) were administered intraperitoneally (IP).

Treatment Groups:

Single compound groups received either amonafide (60 mg/kg), caffeine (75 mg/kg), or nicotinamide (1000 mg/kg).

Combination groups received amonafide (60 mg/kg), in combination with either caffeine (75 mg/kg), or nicotinamide (1000 mg/kg).

Sequential treatment groups had a 1 hr interval between administration of the compounds. All compounds were administered at the same concentration as the combination groups (amonafide (60 mg/kg), caffeine (75 mg/kg), nicotinamide (1000 mg/kg). Sequential groups included one group treated with amonafide followed by treatment with caffeine, one group treated with caffeine followed by treatment with amonafide, one group treated with amonafide followed by treatment with nicotinamide, and one group treated with nicotinamide followed by treatment with amonafide.

Four mice were used in each treatment group.

Analysis

Tumors were measured 3 times a week using Vernier calipers, and tumor volume (V) was calculated according to the formula:

$$V = \frac{π}{6} \times {D}_{1} \times {D}_{2} \times {D}_{3}$$

Where D₁₋₃ are perpendicular diameters measured in millimeters (mm).

Tumor volume quadrupling time was defined as the time (days) for treated and untreated tumors to grow to four times (4×) their initial treatment volume. Tumor growth delay ratio (T/C) was defined as the ratio of 4× growth times of treated (T) and untreated control (C) tumors.

Results

TABLE 1

| | # of | Dose | Days to 4× | | | |
|---|---|---|---|---|---|---|
| Treatment | Tumors | (mg/kg) | (Ave ± SE) | T/C | Median | Delay |
| Untreated | 8 | — | 4.8 ± 0.3 | — | 4.5 | — |
| Amonafide | 8 | 60 | 7.6 ± 0.4 | 1.6 | 7.5 | 2.97 |
| Caffeine | 8 | 75 | 6.0 ± 0.5 | 1.2 | 5.5 | 1.01 |
| Combination | 6 of 8 | 60/75 | 6.2 ± 0.4 | 1.3 | 5.8 | 1.29 |
| Amonafide and Caffeine | | | | | | |
| Sequential | 8 | 60/75 | 8.9 ± 0.5 | 1.8 | 9.2 | 4.67 |
| Amonafide→ Caffeine | | | | | | |
| Sequential | 8 | 75/60 | 8.6 ± 0.7 | 1.8 | 8.4 | 3.88 |
| Caffeine→ Amonafide | | | | | | |
| Nicotinamide | 8 | 1000 | 5.4 ± 0.3 | 1.1 | 5.3 | 0.76 |
| Combination | 6 of 8 | 60/1000 | 8.3 ± 0.9 | 1.7 | 7.2 | 2.63 |
| Amonafide and | | | | | | |
| Nicotinamide | | | | | | |
| Sequential | 8 | 60/1000 | 8.7 ± 1.0 | 1.8 | 7.8 | 3.28 |
| Amonafide→ Nicotinamide | | | | | | |
| Sequential | 8 | 1000/60 | 8.7 ± 1.0 | 1.8 | 8.3 | 3.76 |
| Nicotinamide→ Amonafide | | | | | | |

As shown in Table 1 and in FIGS. 7 and 8, PARP-1 inhibitors increased the tumor growth delay of amonafide in the RIF-1 murine model.

Amonafide and Caffeine

Caffeine given sequentially with amonafide increases the tumor growth delay as compared to treatment with amonafide alone.

Amonafide and Nicotinamide

Nicotinamide given sequentially and in combination with amonafide increases the tumor growth delay as compared to treatment with amonafide alone.

### Example 2

The effects of the PARP-1 inhibitors, caffeine and nicotinamide, on the efficacy of amonafide treatment in RIF-1 cells were determined as discussed below.

Experimental Details

Compounds:

Naphthalimide-Amonafide dihydrochloride (Quinamed®, AMF), substituted naphthalimide soluble in saline at therapeutic concentrations.

PARP-1 inhibitors—caffeine and nicotinamide.

In vitro Model:

RIF-1 cells were plated in a 96 well plate, 40K cells/well for test wells. Cell dilutions for controls: 5K, 10K, 20K & 40K cells/well.

Treatment Groups:

Single compound groups received either amonafide at 10 uM, 50 uM and 100 uM, caffeine at 1000 uM, or nicotinamide at 10 uM and 100 uM.

Combination groups received amonafide (10 uM) in combination with either caffeine(1000 uM) or nicotinamide (10 uM).

Sequential treatment groups had a 3 hr interval between administrations of the compounds. All compounds were administered at the same concentration as the combination groups (amonafide (10 uM), caffeine (1000 uM), nicotinamide (10 uM)). Sequential groups included one group treated with amonafide followed by treatment with caffeine, one group treated with caffeine followed by treatment with amonafide, one group treated with amonafide followed by treatment with nicotinamide, and one group treated with nicotinamide followed by treatment with amonafide.

Analysis

Viability was accessed using alamarBlue, measured 24 hr. after the addition of the first drug treatment. Ahmed, S. A. ,et al., J. Immunol Methods 170(2):211-224 (1994).

Results

As shown FIG. 9, PARP-1 inhibitors increase the growth inhibition of amonafide in a 24 hour continuous exposure treatment in vitro cell viability assay.

Amonafide and Caffeine

Caffeine given sequentially and in combination with amonafide increases the growth inhibition of RIF-1 cells as compared to treatment with amonafide alone.

Amonafide and Nicotinamide

Nicotinamide given sequentially and in combination with amonafide increases the growth inhibition of RIF-1 cells as compared to treatment with amonafide alone.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the claims appended hereto and their equivalents. All references presented herein are expressly incorporated by reference.

1. A method of treating a cellular proliferative disease, comprising administering to a patient in need thereof a naphthalimide and a poly-(ADP ribose) polymerase-1 (PARP-1) inhibitor.

2. The method according to claim 1, wherein said naphthalimide comprises amonafide.

3. The method according to claim 1, wherein said PARP-1 inhibitor comprises caffeine.

4. The method according to claim 1, wherein said PARP-1 inhibitor comprises nicotinamide.

5. The method according to claim 1, wherein said naphthalimide comprises an amonafide analog.

6. The method according to claim 1 wherein said patient is a human.

7. The method according to claim 1 wherein said naphthalimide is administered in combination with said PARP-1 inhibitor.

8. The method according to claim 1 wherein said naphthalimide is administered sequentially with said PARP-1 inhibitor.

9. The method according to claim 1 wherein said cellular proliferative disease is a solid tumor.

10. The method according to claim 1 wherein said cellular proliferative disease is prostate cancer.

11. The method according to claim 1 wherein said cellular proliferative disease is breast cancer.
